(19) United States
(12) Patent Application Publication
(10) Pub. No.: US 2020/0045112 A1
(43) Pub. Date: Feb. 6, 2020

(54) MEDICAL IMAGING DEVICE MESSAGING SERVICE

(71) Applicant: Hyperfine Research, Inc., Guilford, CT (US)

(72) Inventors: Laura Sacolick, Guilford, CT (US); Michael Stephen Poole, Guilford, CT (US); Arjun Sadanand, New Haven, CT (US); Edward Welch, Guilford, CT (US)

(21) Appl. No.: 16/524,516

(22) Filed: Jul. 29, 2019

Related U.S. Application Data
(60) Provisional application No. 62/712,636, filed on Jul. 31, 2018.

(51) Int. Cl. H04L 29/08; G01R 33/54; G01R 33/44; G16H 30/20; G16H 40/67

(52) U.S. Cl. CPC H04L 67/12; G01R 33/546; G01R 33/543; H04L 51/22; H04L 67/025; G16H 30/20; G16H 40/67; G01R 33/445

(57) ABSTRACT

A system and method for operating a magnetic resonance imaging system including a magnetics system and a controller located in a same room as the magnetics system and communicatively coupled to at least one communication network. The method includes operating the magnetic resonance system to acquire at least one magnetic resonance image of a patient, and, in response to a triggering event, transmitting, via the at least one communication network, a message including metadata associated with acquisition of the at least one magnetic resonance image and/or results thereof to one or more recipients.

### CROSS-REFERENCE TO RELATED APPLICATIONS

The present application claims the benefit under 35 U.S.C. § 119(e) of U.S. Provisional Patent Application No. 62/712,636, filed Jul. 31, 2018, titled “Medical Imaging Device Messaging Service,” which is hereby incorporated by reference in its entirety.

### BACKGROUND

Magnetic resonance imaging (MRI) provides an important imaging modality for numerous applications and is widely utilized in clinical and research settings to produce images of the inside of the human body. However, there are a number of drawbacks to MRI that, for a given imaging application, may involve the relatively high cost of the equipment, limited availability and/or difficulty in gaining access to clinical MRI scanners and/or the length of the image acquisition process.

To receive an MRI, a patient may schedule an MRI examination far in advance and/or travel a distance to a specialized facility. Since the scheduled time for the MRI is known, the patient's doctor may attempt access the generated MR images sometime after the scheduled time for the MRI exam has passed.

### SUMMARY

Some embodiments are directed to a magnetic resonance imaging system. The magnetic resonance imaging system comprises a magnetics system having a plurality of magnetics components configured to produce magnetic fields to perform magnetic resonance imaging, the plurality of magnetics components comprising at least one magnetics component configured to produce a B₀ magnetic field; and a controller communicatively coupled to at least one communication network and configured to control the magnetics system to acquire at least one magnetic resonance image of a patient; and in response to a triggering event, transmit, via the at least one communication network, a message comprising metadata associated with acquisition of the at least one magnetic resonance image and/or results thereof to one or more recipients.

Some embodiments are directed to a method of controlling a magnetic resonance imaging system, the magnetic resonance system comprising a magnetics system having a plurality of magnetics components configured to produce magnetic fields to perform magnetic resonance imaging. The method comprises using a controller communicatively coupled to at least one communication network to control the magnetic resonance system to acquire at least one magnetic resonance image of a patient; and, in response to a triggering event transmit, via the at least one communication network, a message comprising metadata associated with acquisition of the at least one magnetic resonance image and/or results thereof to one or more recipients. Some embodiments are directed to an at least one non-transitory computer-readable storage medium storing processor-executable instructions that, when executed by a magnetic resonance imaging (MRI) system, cause the MRI system to perform a method. The method comprises using a controller communicatively coupled to at least one communication network to: control the MRI system to acquire a magnetic resonance (MR) image of a patient; and in response to a triggering event: transmit, via the at least one communication network, a message comprising metadata associated with acquisition of the MR image and/or the MR image to one or more recipients.

In some embodiments, the controller is located in a same room as the magnetic resonance imaging system.

In some embodiments, the message comprises an email, a short message service (SMS), or a multimedia messaging service (MMS).

In some embodiments, the method further comprises removing confidential patient information from the metadata associated with acquisition of the at least one magnetic resonance image prior to transmitting the message.

In some embodiments, the metadata associated with acquisition of the at least one magnetic resonance image comprises information about the patient, information about the magnetic resonance imaging protocol associated with acquisition of the at least one magnetic resonance image, information identifying an operator of the magnetic resonance imaging system and/or contact information associated with the operator, and/or information identifying the physical location of the magnetic resonance imaging system.

In some embodiments, the metadata associated with acquisition of the at least one magnetic resonance image comprises a hyperlink to a web-based magnetic resonance image viewing software program and/or a hyperlink to an interface for remote operation of the magnetic resonance imaging system.

In some embodiments, the triggering event comprises input received from an operator of the magnetic resonance imaging system.

In some embodiments, the triggering event comprises, while acquiring a plurality of magnetic resonance images, acquisition of one magnetic resonance image of the plurality of magnetic resonance images and/or acquisition of the last magnetic resonance image of the plurality of magnetic resonance images.

In some embodiments, the magnetics system comprises a B₀ magnet comprising a permanent magnet.

In some embodiments, the magnetics system comprises a B₀ magnet configured to produce a B₀ magnetic field having a field strength equal to or less than approximately 0.2 T and greater than or equal to approximately 20 mT.

In some embodiments, the magnetics system comprises a B₀ magnet configured to produce a B₀ magnetic field having a field strength equal to or less than approximately 1 T and greater than or equal to approximately 50 mT.

In some embodiments, the magnetics system comprises a B₀ magnet configured to produce a B₀ magnetic field having a field strength greater than or equal to approximately 1 T. In some embodiments, the magnetics system comprises a B₀ magnet configured to produce a B₀ magnetic field having a field strength equal to or less than approximately 7 T and greater than or equal to approximately 1 T.

In some embodiments, the magnetic resonance imaging system is configured to be operated in an unshielded room.

In some embodiments, the magnetic resonance imaging system further comprises a conveyance mechanism to allow the magnetic resonance imaging system to be moved to desired locations.

Some embodiments are directed to a medical imaging device. The medical imaging device comprises a controller, communicatively coupled to at least one communication network, and configured to control the medical imaging device to acquire a medical image of a patient; and in response to a triggering event, transmit, via the at least one communication network, a message comprising metadata associated with acquisition of the medical image and/or the medical image to one or more recipients.

Some embodiments are directed to a method of operating a medical imaging device. The method comprises using a controller communicatively coupled to at least one communication network to control the medical imaging device to acquire a medical image of a patient; and in response to a triggering event, transmit, via the at least one communication network, a message comprising metadata associated with acquisition of the medical image and/or the medical image to one or more recipients.

Some embodiments are directed to at least one non-transitory computer-readable storage medium storing processor-executable instructions that, when executed by a medical imaging device, cause the at least one medical imaging device to perform a method. The method comprises using a controller communicatively coupled to at least one communication network to control the medical imaging device to acquire a medical image of a patient; and in response to a triggering event, transmit, via the at least one communication network, a message comprising metadata associated with acquisition of the medical image and/or the medical image to one or more recipients.

In some embodiments, the medical imaging device comprises an ultrasound imaging device.

In some embodiments, the medical imaging device comprises a computed tomography (CT) imaging device.

In some embodiments, the medical imaging device comprises a positron emission tomography (PET) imaging device.

In some embodiments, the medical imaging device comprises a single-photon emission computerized tomography (SPECT) imaging device.

In some embodiments, the medical imaging device comprises an X-ray imaging device.

In some embodiments, the medical imaging device comprises a magnetic resonance imaging (MRI) device.

In some embodiments, the message comprises an email, a short message service (SMS), and/or a multimedia messaging service (MMS).

In some embodiments, the method further comprises removing confidential patient information from the metadata associated with acquisition of the medical image prior to transmitting the message.

In some embodiments, the metadata associated with acquisition of the medical image comprises information about the patient.

In some embodiments, the metadata associated with acquisition of the medical image comprises information about the MRI protocol associated with acquisition of the medical image.

In some embodiments, the metadata associated with acquisition of the medical image comprises information identifying an operator of the medical imaging device and/or contact information associated with the operator.

In some embodiments, the metadata associated with acquisition of the medical image comprises information identifying the physical location of the medical imaging device.

In some embodiments, the metadata associated with acquisition of the medical image comprises a hyperlink to a web-based medical image viewing software program.

In some embodiments, the metadata associated with acquisition of the medical image comprises a hyperlink to an interface for remote operation of the medical imaging device.

In some embodiments, the triggering event comprises input received from an operator of the medical imaging device.

In some embodiments, the triggering event comprises completion of acquisition of the medical image.

In some embodiments, the triggering event comprises start of acquisition of the medical image.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale.

FIG. 1 illustrates exemplary components of a magnetic resonance imaging system, in accordance with some embodiments;

FIG. 2 illustrates a B₀ magnet comprising a plurality of permanent magnets that may be part of the MRI system of FIG. 1, in accordance with some embodiments;

FIGS. 3A and 3B illustrate views of a portable MRI system, in accordance with some embodiments;

FIG. 3C illustrates another example of a portable MRI system, in accordance with some embodiments;

FIG. 4 illustrates a portable MRI system performing a scan of a patient's head, in accordance with some embodiments;

FIG. 5 illustrates an exemplary system for implementing a messaging service, in accordance with some embodiments;

FIGS. 6A, 6B, and 6C illustrate a user interface of a messaging service, in accordance with some embodiments;

FIGS. 7A and 7B illustrate an exemplary message sent by a messaging service, in accordance with some embodiments;

FIG. 8 is a flowchart of an illustrative process for sending a message using a messaging service, in accordance with some embodiments; and

FIG. 9 shows, schematically, an illustrative computer 900 on which any aspect of the technology described herein may be implemented.

### DETAILED DESCRIPTION

As described above, conventional high-field MRI examinations are often scheduled in advance because of their limited availability and high cost. When such examinations are scheduled, a patient's medical care team will know when to expect results from the MRI examination. However, the deployment and use of a portable, low-field MRI system allows for unscheduled examinations, emergency imaging procedures, or periodic monitoring of a patient over a period of time. The inventors have recognized that no solutions exist for coordinating the analysis and communication of such unscheduled imaging results across a patient's medical team, which can consist of multiple physicians, nurses, technicians, etc.

Conventional MRI can be improved by providing data to a patient's medical team as soon as it is made available by the MRI system. For example, when monitoring a patient's condition over a period of time, it can be helpful for the patient's medical team to receive messages from the MRI system periodically during the monitoring and/or in case of a status change of the patient. Such rapid messaging can enable a faster response from a patient's medical team in case of an emergency (e.g., detection of internal bleeding, etc.) and/or any other change in the patient's condition that warrants notifying the patient's medical team.

However, conventional MRI systems do not transmit MRI image data or associated metadata to a patient's medical team. Because conventional MRI systems operate in high field regimes, they are deployed in shielded rooms and transmit raw MR signal reads, via shielded cabling, to a control console located in a separate room from the one in which the MRI system is housed. The MR signal reads, which may constitute a series of values corresponding to spatial frequency domain (k-space) measurements, are then processed by the control console to generate an MR image. In turn, the MR image may be viewed by members of the patient's medical team at the control console. Such conventional installations do not allow providing the patient's medical team with imaging results and associated information in real-time.

The inventors have appreciated that a low-field MRI system, which operates at lower magnetic field strengths than a conventional MRI system and with lower environmental electromagnetic noise limitations, is not limited to being operated in a shielded room. For example, the low-field MRI system developed by the Assignee of this application and described in U.S. Pat. No. 10,274,561 filed Jan. 24, 2018 and titled “Electromagnetic Shielding for Magnetic Resonance Imaging Methods and Apparatus,” which is incorporated by reference herein in its entirety, is not limited to being operated in a shielded room. Accordingly, the inventors have developed a system for sending, to one or more members of the patient's medical team, message directly from the MRI system responsive to predefined trigger events. The messages sent by the MRI system include complete magnetic resonance (MR) images as well as metadata associated with the MR images (e.g., information about the protocol, time of the examination, etc.), as will be described below.

The inventors have developed a system for sending messages containing metadata associated with an MRI examination and/or magnetic resonance images directly from an imaging device to one or more medical professionals and/or other people associated with a patient. The messaging service provides information to the medical professional(s) and, in some embodiments, allows the medical professional(s) to provide responsive input (e.g., by text, email, chat session, etc.). In some embodiments, a message may be an e-mail notification, an SMS message, an MMS message, a phone message, an instance message via an instant messaging service, a message over a chat service, a message through any suitable service and/or protocol, etc., and/or any other suitable type of message.

In some embodiments, an operator of a medical imaging device may specify a group of one or more people to be notified when the medical imaging device obtains one or more medical images of a patient (e.g., after completing scanning a patient using a magnetic resonance imaging or other medical imaging scanner). The list of people to be notified may include one or more physicians, one or more radiologists, one or more nurses, and/or one or more other medical professionals associated with the patient.

In some embodiments, the medical imaging device may message one or more people on the list and provide them with a message that includes medical images and any associated data (e.g., magnetic resonance images and associated data) as soon as the medical images are available. The messaging service may also be used to request that one or more people in the list go in person to the patient being imaged. In some embodiments, the messaging service may send images to one or more people on this list during and/or after medical exams so that the people may review the images for artifacts, patient positioning, and contrast protocol. In some embodiments, the messaging service may provide one or more people on the list with a hyperlink to join a live scanning session. They can check images for major problems or changes in the patient's medical state. They also can reply back either with messages that get shown on the scanner interface, or join a live scanning session.

The messaging service developed by the inventors may be used in conjunction with numerous types of medical imaging devices including, but not limited to, ultrasound imaging devices, computed tomography (CT) imaging devices, positron emission tomography (PET) imaging devices, single-photon emission computerized tomography (SPECT) imaging devices, X-ray imaging devices, magnetic resonance imaging (MRI) devices, portable MRI devices, and low-field MRI imaging devices including any of the MR imaging devices described in in U.S. Pat. App. Pub. No. 2018/0164390, titled “Electromagnetic Shielding for Magnetic Resonance Imaging Methods and Apparatus,” which is incorporated by reference herein in its entirety. As used herein, “high-field” refers generally to MRI systems presently in use in a clinical setting and, more particularly, to MRI systems operating with a main magnetic field (i.e., a B₀ field) at or above 1.5 T, though clinical systems operating between 0.5 T and 1.5 T are typically also considered “high-field.” By contrast, “low-field” refers generally to MRI systems operating with a B₀ field of less than or equal to approximately 0.2 T.

In some embodiments, an operator of a medical imaging device can specify one or more message service message recipients by entering e-mail addresses (or other types of identifiers) for each individual, creating group lists, accessing previously-specified group lists, and/or specifying previously-created lists of prior recipients (e.g., for a previous message).

In some embodiments, a message sent to a recipient by the messaging service may be sent at the end of a patient exam. In some embodiments, a message may be sent after every imaging scan is completed. In some embodiments, a message may be sent when triggered by an operator of a medical imaging device during or after an exam of a patient. In some embodiments, a message may be sent when triggered by a change in a patient's imaging results while monitoring the patient over a period of time.

In some embodiments, a message from an imaging device (and/or a computer coupled to or otherwise associated with the imaging device) to a recipient may include one or more of the following items: one or more medical images, one or more reconstructed images, one or more post-processed images, one or more composite images including one or more annotations, one or more values derived from one or more images, one or more overlays or other data derived from the original scan data, one or more detected changes, one or more segmentations, one or more registrations to atlases, one or more diagnostic aids output from any suitable post-processing algorithm, one or more image files, an embedded viewer (e.g., DICOM viewer), one or more links to an image on a patient archiving communication system (PACS), information identifying a patient (e.g., name, date of birth, identifying number, sex, indication, etc.), exam information (date, time, location, protocol, read urgency etc.), scan information (sequence type, contrast information, resolution, etc.), status of exam (error, problem reports, indicator of success/failure), free-form comments, one or more links to a user interface for the imaging device over web server to log in live to the scanning session, one or more links to an mobile computing device (e.g., iPad) camera, and/or any other suitable information.

Following below are more detailed descriptions of various concepts related to, and embodiments of, techniques for automatic messaging. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

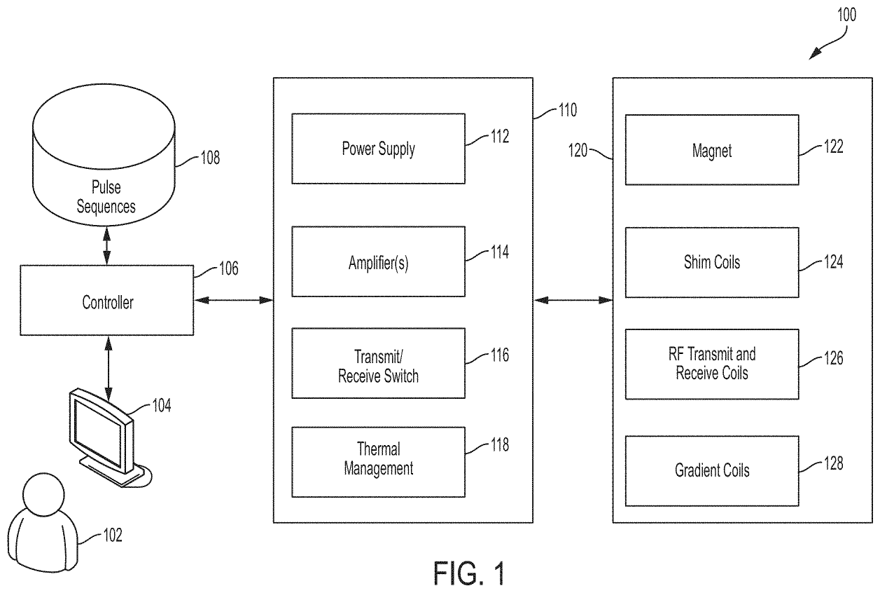

FIG. 1 is a block diagram of typical components of a MRI system 100. In the illustrative example of FIG. 1, MRI system 100 comprises computing device 104, controller 106, pulse sequences store 108, power management system 110, and magnetics components 120. It should be appreciated that system 100 is illustrative and that a MRI system may have one or more other components of any suitable type in addition to or instead of the components illustrated in FIG. 1. However, a MRI system will generally include these high level components, though the implementation of these components for a particular MRI system may differ vastly, as described in further detail below.

As illustrated in FIG. 1, magnetics components 120 comprise B₀ magnet 122, shim coils 124, RF transmit and receive coils 126, and gradient coils 128. Magnet 122 may be used to generate the main magnetic field B₀. Magnet 122 may be any suitable type or combination of magnetics components that can generate a desired main magnetic B₀ field. As described above, in the high field regime, the B₀ magnet is typically formed using superconducting material generally provided in a solenoid geometry, requiring cryogenic cooling systems to keep the B₀ magnet in a superconducting state. Thus, high-field B₀ magnets are expensive, complicated and consume large amounts of power (e.g., cryogenic cooling systems require significant power to maintain the extremely low temperatures needed to keep the B₀ magnet in a superconducting state), require large dedicated spaces, and specialized, dedicated power connections (e.g., a dedicated three-phase power connection to the power grid). Conventional low-field B₀ magnets (e.g., B₀ magnets operating at 0.2 T) are also often implemented using superconducting material and therefore have these same general requirements. Other conventional low-field B₀ magnets are implemented using permanent magnets, which to produce the field strengths to which conventional low-field systems are limited (e.g., between 0.2 T and 0.3 T due to the inability to acquire useful images at lower field strengths), need to be very large magnets weighing 5-20 tons. Thus, the B₀ magnet of conventional MRI systems alone prevents both portability and affordability.

Gradient coils 128 may be arranged to provide gradient fields and, for example, may be arranged to generate gradients in the B₀ field in three substantially orthogonal directions (X, Y, Z). Gradient coils 128 may be configured to encode emitted MR signals by systematically varying the B₀ field (the B₀ field generated by magnet 122 and/or shim coils 124) to encode the spatial location of received MR signals as a function of frequency or phase. For example, gradient coils 128 may be configured to vary frequency or phase as a linear function of spatial location along a particular direction, although more complex spatial encoding profiles may also be provided by using nonlinear gradient coils. For example, a first gradient coil may be configured to selectively vary the B₀ field in a first (X) direction to perform frequency encoding in that direction, a second gradient coil may be configured to selectively vary the B₀ field in a second (Y) direction substantially orthogonal to the first direction to perform phase encoding, and a third gradient coil may be configured to selectively vary the B₀ field in a third (Z) direction substantially orthogonal to the first and second directions to enable slice selection for volumetric imaging applications. As described above, conventional gradient coils also consume significant power, typically operated by large, expensive gradient power sources, as described in further detail below.

MRI is performed by exciting and detecting emitted MR signals using transmit and receive coils, respectively (often referred to as radio frequency (RF) coils). Transmit/receive coils may include separate coils for transmitting and receiving, multiple coils for transmitting and/or receiving, or the same coils for transmitting and receiving. Thus, a transmit/receive component may include one or more coils for transmitting, one or more coils for receiving and/or one or more coils for transmitting and receiving. Transmit/receive coils are also often referred to as Tx/Rx or Tx/Rx coils to generically refer to the various configurations for the transmit and receive magnetics component of an MRI system. These terms are used interchangeably herein. In FIG. 1, RF transmit and receive coils 126 comprise one or more transmit coils that may be used to generate RF pulses to induce an oscillating magnetic field B 1. The transmit coil(s) may be configured to generate any suitable types of RF pulses.

Power management system 110 includes electronics to provide operating power to one or more components of the low-field MRI system 100. For example, as described in more detail below, power management system 110 may include one or more power supplies, gradient power components, transmit coil components, and/or any other suitable power electronics needed to provide suitable operating power to energize and operate components of MRI system 100. As illustrated in FIG. 1, power management system 110 comprises power supply 112, power component(s) 114, transmit/receive switch 116, and thermal management components 118 (e.g., cryogenic cooling equipment for superconducting magnets). Power supply 112 includes electronics to provide operating power to magnetic components 120 of the MRI system 100. For example, power supply 112 may include electronics to provide operating power to one or more B₀ coils (e.g., B₀ magnet 122) to produce the main magnetic field for the low-field MRI system. Transmit/receive switch 116 may be used to select whether RF transmit coils or RF receive coils are being operated.

Power component(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and one or more shim power components configured to provide power to one or more shim coils (e.g., shim coils 124).

In conventional MRI systems, the power components are large, expensive and consume significant power. Typically, the power electronics occupy a room separate from the MRI scanner itself. The power electronics not only require substantial space, but are expensive complex devices that consume substantial power and require wall mounted racks to be supported. Thus, the power electronics of conventional MRI systems also prevent portability and affordability of MRI.

As illustrated in FIG. 1, MRI system 100 includes controller 106 (also referred to as a console) having control electronics to send instructions to and receive information from power management system 110. Controller 106 may be configured to implement one or more pulse sequences, which are used to determine the instructions sent to power management system 110 to operate the magnetic components 120 in a desired sequence (e.g., parameters for operating the RF transmit and receive coils 126, parameters for operating gradient coils 128, etc.). As illustrated in FIG. 1, controller 106 also interacts with computing device 104 programmed to process received MR data. For example, computing device 104 may process received MR data to generate one or more MR images using any suitable image reconstruction process(es). Controller 106 may provide information about one or more pulse sequences to computing device 104 for the processing of data by the computing device. For example, controller 106 may provide information about one or more pulse sequences to computing device 104 and the computing device may perform an image reconstruction process based, at least in part, on the provided information. In conventional MRI systems, computing device 104 typically includes one or more high performance work-stations configured to perform computationally expensive processing on MR data relatively rapidly. Such computing devices are relatively expensive equipment on their own.

As should be appreciated from the foregoing, currently available clinical MRI systems (including high-field, mid-field and low-field systems) are large, expensive, fixed installations requiring substantial dedicated and specially designed spaces, as well as dedicated power connections. The inventors have developed low-field, including very-low field, MRI systems that are lower cost, lower power and/or portable, significantly increasing the availability and applicability of MRI. According to some embodiments, a portable MRI system is provided, allowing an MRI system to be brought to the patient and utilized at locations where it is needed.

As described above, some embodiments include an MRI system that is portable, allowing the MRI device to be moved to locations in which it is needed (e.g., emergency and operating rooms, primary care offices, neonatal intensive care units, specialty departments, emergency and mobile transport vehicles and in the field). There are numerous challenges that face the development of a portable MRI system, including size, weight, power consumption and the ability to operate in relatively uncontrolled electromagnetic noise environments (e.g., outside a specially shielded room). As described above, currently available clinical MRI systems range from approximately 4-20 tons. Thus, currently available clinical MRI systems are not portable because of the sheer size and weight of the imaging device itself, let alone the fact that currently available systems also require substantial dedicated space, including a specially shielded room to house the MRI scanner and additional rooms to house the power electronics and the technician control area, respectively. The inventors have developed MRI systems of suitable weight and size to allow the MRI system to be transported to a desired location, some examples of which are described in further detail below.

The weight of the B₀ magnet is a significant portion of the overall weight of the MRI system which, in turn, impacts the portability of the MRI system. In embodiments that primarily use low carbon and/or silicon steel for the yoke and shimming components, an exemplary B₀ magnet 200 dimensioned similar to that described in the foregoing may weigh approximately 550 kilograms. According to some embodiments, cobalt steel (CoFe) may be used as the primary material for the yoke (and possibly the shim components), potentially reducing the weight of B₀ magnet 200 to approximately 450 Kilograms. However, CoFe is generally more expensive than, for example, low carbon steel, driving up the cost of the system. Accordingly, in some embodiments, select components may be formed using CoFe to balance the tradeoff between cost and weight arising from its use. Using such exemplary B₀ magnets a portable, cartable or otherwise transportable MRI system may be constructed, for example, by integrating the B₀ magnet within a housing, frame or other body to which castors, wheels or other means of locomotion can be attached to allow the MRI system to be transported to desired locations (e.g., by manually pushing the MRI system and/or including motorized assistance). As a result, an MRI system can be brought to the location in which it is needed, increasing its availability and use as a clinical instrument and making available MRI applications that were previously not possible. According to some embodiments, the total weight of a portable MRI system is less than 1,500 pounds and, preferably, less than 1000 pounds to facilitate maneuverability of the MRI system.

A further aspect of portability involves the capability of operating the MRI system in a wide variety of locations and environments. As described above, currently available clinical MRI scanners are required to be located in specially shielded rooms to allow for correct operation of the device and is one (among many) of the reasons contributing to the cost, lack of availability and non-portability of currently available clinical MRI scanners. Thus, to operate outside of a specially shielded room and, more particularly, to allow for generally portable, cartable or otherwise transportable MRI, the MRI system must be capable of operation in a variety of noise environments. The inventors have developed noise suppression techniques that allow the MRI system to be operated outside of specially shielded rooms, facilitating both portable/transportable MRI as well as fixed MRI installments that do not require specially shielded rooms. While the noise suppression techniques allow for operation outside specially shielded rooms, these techniques can also be used to perform noise suppression in shielded environments, for example, less expensive, loosely or ad-hoc shielding environments, and can be therefore used in conjunction with an area that has been fitted with limited shielding, as the aspects are not limited in this respect.

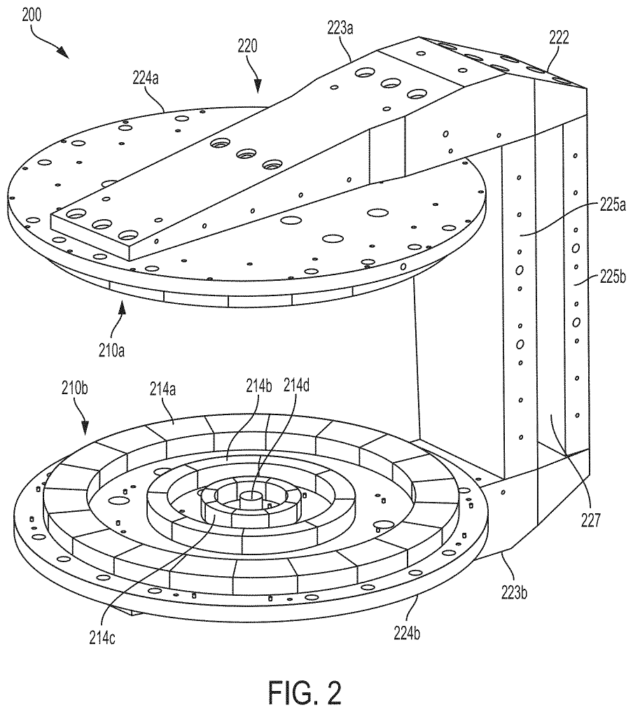

FIG. 2 illustrates a B₀ magnet 200, in accordance with some embodiments. In particular, B₀ magnet 200 is formed by permanent magnets 210a and 210b arranged in a bi-planar geometry with a yoke 220 coupled thereto to capture electromagnetic flux produced by the permanent magnets and transfer the flux to the opposing permanent magnet to increase the flux density between permanent magnets 210a and 210b. Each of permanent magnets 210a and 210b are formed from a plurality of concentric permanent magnets, as shown by permanent magnet 210b comprising an outer ring of permanent magnets 214a, a middle ring of permanent magnets 214b, an inner ring of permanent magnets 214c, and a permanent magnet disk 214d at the center. Permanent magnet 210a may comprise the same set of permanent magnet elements as permanent magnet 210b. The permanent magnet material used may be selected depending on the design requirements of the system (e.g., NdFeB, SmCo, etc. depending on the properties desired).

The permanent magnet material used may be selected depending on the design requirements of the system. For example, according to some embodiments, the permanent magnets (or some portion thereof) may be made of NdFeB, which produces a magnetic field with a relatively high magnetic field per unit volume of material once magnetized. According to some embodiments, SmCo material is used to form the permanent magnets, or some portion thereof. While NdFeB produces higher field strengths (and in general is less expensive than SmCo), SmCo exhibits less thermal drift and thus provides a more stable magnetic field in the face of temperature fluctuations. Other types of permanent magnet material(s) may be used as well, as the aspects are not limited in this respect. In general, the type or types of permanent magnet material utilized will depend, at least in part, on the field strength, temperature stability, weight, cost and/or ease of use requirements of a given B₀ magnet implementation.

The permanent magnet rings are sized and arranged to produce a homogenous field of a desired strength in the central region (field of view) between permanent magnets 210a and 210b. In the exemplary embodiment illustrated in FIG. 2, each permanent magnet ring comprises a plurality of blocks of ferromagnetic material to form the respective ring. The blocks forming each ring may be dimensioned and arranged to produce a desired magnetic field. The inventors have recognized that the blocks may be dimensioned in a number of ways to decrease cost, reduce weight and/or improve the homogeneity of the magnetic field produced, as described in further detail in connection with the exemplary rings that together form permanent magnets of a B₀ magnet, in accordance with some embodiments.

B₀ magnet 200 further comprises yoke 220 configured and arranged to capture magnetic flux generated by permanent magnets 210a and 210b and direct it to the opposing side of the B₀ magnet to increase the flux density in between permanent magnets 210a and 210b, increasing the field strength within the field of view of the B₀ magnet. By capturing magnetic flux and directing it to the region between permanent magnets 210a and 210b, less permanent magnet material can be used to achieve a desired field strength, thus reducing the size, weight and cost of the B₀ magnet. Alternatively, for given permanent magnets, the field strength can be increased, thus improving the SNR of the system without having to use increased amounts of permanent magnet material. For exemplary B₀ magnet 200, yoke 220 comprises a frame 222 and plates 224a and 224b. In a manner similar to that described above in connection with yoke 220, plates 324a and 324b capture magnetic flux generated by permanent magnets 210a and 210b and direct it to frame 222 to be circulated via the magnetic return path of the yoke to increase the flux density in the field of view of the B₀ magnet. Yoke 220 may be constructed of any desired ferromagnetic material, for example, low carbon steel, CoFe and/or silicon steel, etc. to provide the desired magnetic properties for the yoke. According to some embodiments, plates 224a and 224b (and/or frame 222 or portions thereof) may be constructed of silicon steel or the like in areas where the gradient coils could most prevalently induce eddy currents.

Exemplary frame 222 comprises arms 223a and 223b that attach to plates 224a and 224b, respectively, and supports 225a and 225b providing the magnetic return path for the flux generated by the permanent magnets. The arms are generally designed to reduce the amount of material needed to support the permanent magnets while providing sufficient cross-section for the return path for the magnetic flux generated by the permanent magnets. Arms 223a and 223b have two supports within a magnetic return path for the B₀ field produced by the B₀ magnet. Supports 225a and 225b are produced with a gap 227 formed between, providing a measure of stability to the frame and/or lightness to the structure while providing sufficient cross-section for the magnetic flux generated by the permanent magnets. For example, the cross-section needed for the return path of the magnetic flux can be divided between the two support structures, thus providing a sufficient return path while increasing the structural integrity of the frame. It should be appreciated that additional supports may be added to the structure, as the technique is not limited for use with only two supports and any particular number of multiple support structures.

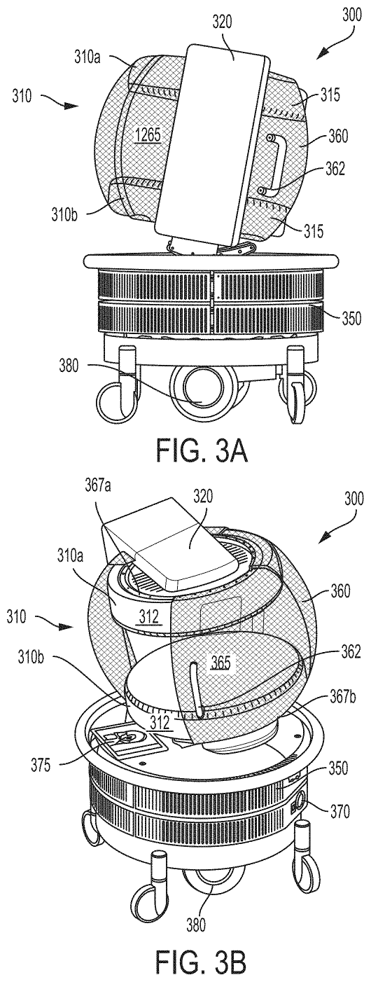

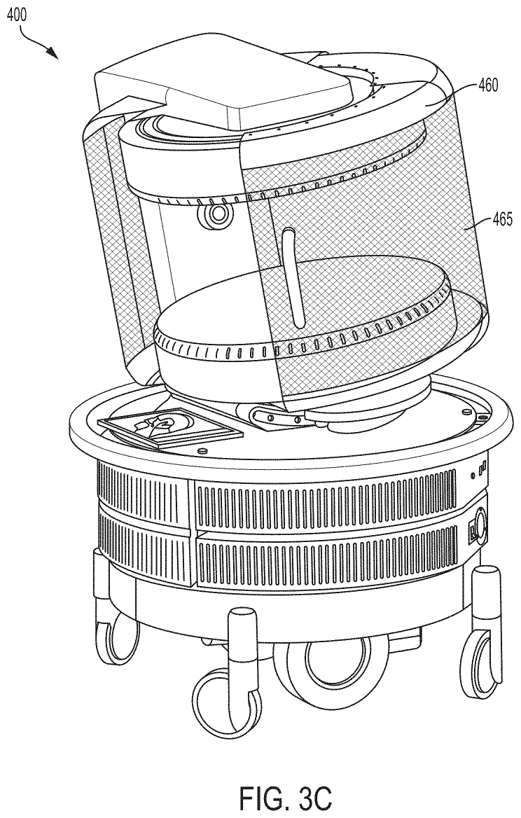

Using the techniques described herein, the inventors have developed portable, low power MRI systems capable of being brought to the patient, providing affordable and widely deployable MRI where it is needed. FIGS. 3A and 3B illustrate views of a portable MRI system, in accordance with some embodiments. Portable MRI system 300 comprises a B₀ magnet 310 formed in part by an upper magnet 310a and a lower magnet 310b having a yoke 320 coupled thereto to increase the flux density within the imaging region. The B₀ magnet 310 may be housed in magnet housing 312 along with gradient coils 315 (e.g., any of the gradient coils described in U.S. application Ser. No. 14/845,652, titled “Low Field Magnetic Resonance Imaging Methods and Apparatus” and filed on Sep. 4, 2015, which is herein incorporated by reference in its entirety). According to some embodiments, B₀ magnet 310 comprises an electromagnet. According to some embodiments, B₀ magnet 310 comprises a permanent magnet, for example, a permanent magnet similar to or the same as permanent magnet 200 illustrated in FIG. 2.

Portable MRI system 300 further comprises a base 350 housing the electronics needed to operate the MRI system. For example, base 350 may house electronics including power components configured to operate the MRI system using mains electricity (e.g., via a connection to a standard wall outlet and/or a large appliance outlet). For example, base 370 may house low power components, such as those described herein, enabling at least in part the portable MRI system to be powered from readily available wall outlets. Accordingly, portable MRI system 300 can be brought to the patient and plugged into a wall outlet in the vicinity.

Portable MRI system 300 further comprises moveable slides 360 that can be opened and closed and positioned in a variety of configurations. Slides 360 include electromagnetic shielding 365, which can be made from any suitable conductive or magnetic material, to form a moveable shield to attenuate electromagnetic noise in the operating environment of the portable MRI system to shield the imaging region from at least some electromagnetic noise. As used herein, the term electromagnetic shielding refers to conductive or magnetic material configured to attenuate the electromagnetic field in a spectrum of interest and positioned or arranged to shield a space, object and/or component of interest. In the context of an MRI system, electromagnetic shielding may be used to shield electronic components (e.g., power components, cables, etc.) of the MRI system, to shield the imaging region (e.g., the field of view) of the MRI system, or both.

The degree of attenuation achieved from electromagnetic shielding depends on a number of factors including the type of material used, the material thickness, the frequency spectrum for which electromagnetic shielding is desired or required, the size and shape of apertures in the electromagnetic shielding (e.g., the size of the spaces in a conductive mesh, the size of unshielded portions or gaps in the shielding, etc.) and/or the orientation of apertures relative to an incident electromagnetic field. Thus, electromagnetic shielding refers generally to any conductive or magnetic barrier that acts to attenuate at least some electromagnetic radiation and that is positioned to at least partially shield a given space, object or component by attenuating the at least some electromagnetic radiation.

It should be appreciated that the frequency spectrum for which shielding (attenuation of an electromagnetic field) is desired may differ depending on what is being shielded. For example, electromagnetic shielding for certain electronic components may be configured to attenuate different frequencies than electromagnetic shielding for the imaging region of the MRI system. Regarding the imaging region, the spectrum of interest includes frequencies which influence, impact and/or degrade the ability of the MRI system to excite and detect an MR response. In general, the spectrum of interest for the imaging region of an MRI system correspond to the frequencies about the nominal operating frequency (i.e., the Larmor frequency) at a given B₀ magnetic field strength for which the receive system is configured to or capable of detecting. This spectrum is referred to herein as the operating spectrum for the MRI system. Thus, electromagnetic shielding that provides shielding for the operating spectrum refers to conductive or magnetic material arranged or positioned to attenuate frequencies at least within the operating spectrum for at least a portion of an imaging region of the MRI system.

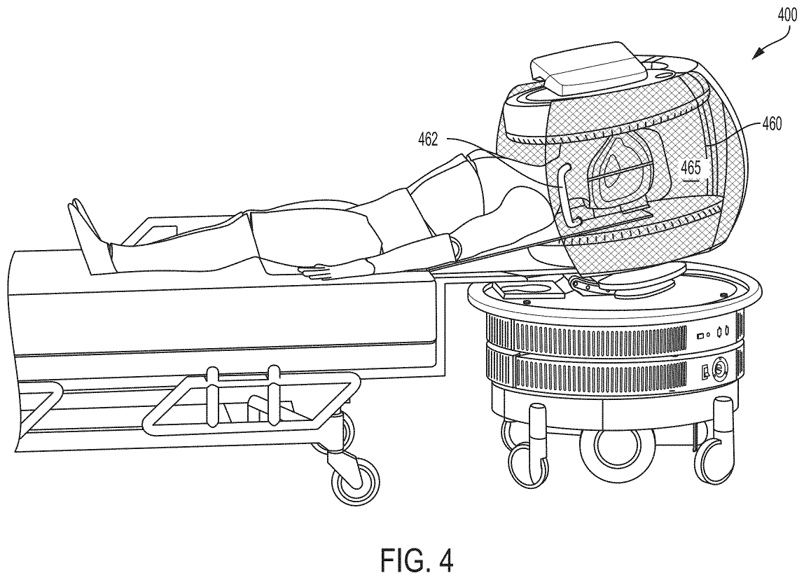

In portable MRI system 300 illustrated, the moveable shields are thus configurable to provide shielding in different arrangements, which can be adjusted as needed to accommodate a patient, provide access to a patient and/or in accordance with a given imaging protocol. For example, for the imaging procedure illustrated in FIG. 4 (e.g., a brain scan), once the patient has been positioned, slides 460 can be closed, for example, using handle 462 to provide electromagnetic shielding 465 around the imaging region except for the opening that accommodates the patient's upper torso. Accordingly, moveable shields allow the shielding to be configured in arrangements suitable for the imaging procedure and to facilitate positioning the patient appropriately within the imaging region.

To ensure that the moveable shields provide shielding regardless of the arrangements in which the slides are placed, electrical gaskets may be arranged to provide continuous shielding along the periphery of the moveable shield. For example, as shown in FIG. 3B, electrical gaskets 367a and 367b may be provided at the interface between slides 360 and magnet housing to maintain to provide continuous shielding along this interface. According to some embodiments, the electrical gaskets are beryllium fingers or beryllium-copper fingers, or the like (e.g., aluminum gaskets), that maintain electrical connection between shields 365 and ground during and after slides 360 are moved to desired positions about the imaging region. According to some embodiments, electrical gaskets 367c are provided at the interface between slides 360 so that continuous shielding is provided between slides in arrangements in which the slides are brought together. Accordingly, moveable slides 360 can provide configurable shielding for the portable MRI system.

FIG. 3C illustrates another example of a portable MRI system, in accordance with some embodiments. Portable MRI system 400 may be similar in many respects to portable MRI systems illustrated in FIGS. 3A and 3B. However, slides 460 are constructed differently, as is shielding 465, resulting in electromagnetic shields that are easier and less expensive to manufacture. As described above, a noise reduction system may be used to allow operation of a portable MRI system in unshielded rooms and with varying degrees of shielding about the imaging region on the system itself, including no, or substantially no, device-level electromagnetic shields for the imaging region. Exemplary shielding designs and noise reduction techniques developed by the inventors are described in U.S. Patent Application Pub. No. 2018/0168527, filed Jan. 24, 2018 and titled “Portable Magnetic Resonance Imaging Methods and Apparatus,” which is herein incorporated by reference in its entirety.

To facilitate transportation, a motorized component 380 is provide to allow portable MRI system to be driven from location to location, for example, using a control such as a joystick or other control mechanism provided on or remote from the MRI system. In this manner, portable MRI system 300 can be transported to the patient and maneuvered to the bedside to perform imaging, as illustrated in FIG. 4. As described above, FIG. 4 illustrates a portable MRI system 400 that has been transported to a patient's bedside to perform a brain scan.

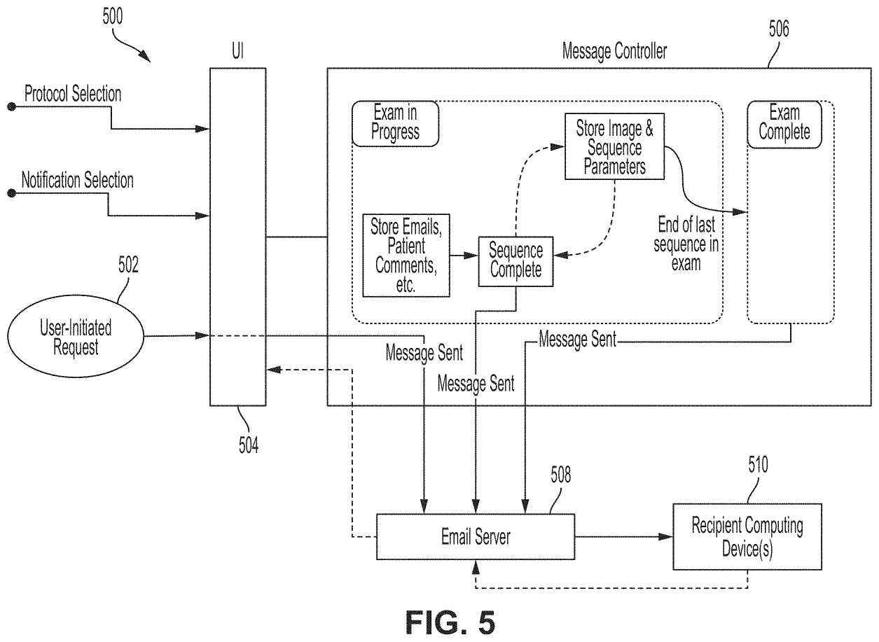

FIG. 5 is a diagram of an illustrative system 500 for implementing a messaging service for a medical imaging device (e.g., an ultrasound imaging device, a computed tomography (CT) imaging device, a positron emission tomography (PET) imaging device, a single-photon emission computerized tomography (SPECT) imaging device, an X-ray imaging device, and/or an MRI system), in accordance with some embodiments of the technology described herein. System 500 may be configured to create and send messages using information obtained from the medical imaging devices. In some embodiments, system 500 may be implemented using hardware (e.g., using an ASIC, an FPGA, or any other suitable circuitry), software (e.g., by executing the software using one or more computer processors), or any suitable combination thereof.

In some embodiments, the messages may be sent as, for example, a short message service (SMS), a multimedia messaging service (MMS), and/or an email. The messages may be sent to one or more recipients, including groups of recipients (e.g., from a pre-selected or newly created lists of emails). The recipients may be selected by an operator of the system 500. The recipients may alternatively or additionally be selected by the system 500 automatically (e.g., based on time of day, based on type of image being acquired).

In some embodiments, system 500 may be configured to send messages in response to triggering events. For example, system 500 may be configured to send a message in response to receiving an input from the user of the medical imaging device. An input may be, for example, a user interaction with a selection area in a user interface. A user interaction may be, for example, a user using a mouse to click a selection area, a user touching a selection area on a touch screen, and/or typing instructions in a selection area. A selection area may be, for example, a button, a slider, a drop down menu, and/or an area to enter text.

As another example, system 500 may be configured to send a message in response to an automatically generated triggering event rather than in response to input provided by the user. For example, a triggering event may be the start of an image acquisition process. For example, the start of the image acquisition process may comprise starting to obtain magnetic resonance (MR) measurements from the patient. Additionally and/or alternatively, the triggering event may be the completion of an image acquisition process. As an example, the completion of an image acquisition process may comprise generating an MR image of the patient from the MR measurements.

In some embodiments, when monitoring a patient, a triggering event may be the passage of a periodic amount of time. For example, the system 500 may be configured to send a message every 10 minutes, every 20 minutes, every 30 minutes, and/or every hour to monitor the patient. Additionally, when monitoring a patient, a triggering event may be a change in the patient's status. For example, the system 500 may be configured to send a message in response to a change in the patient's vital signs. Alternatively and/or additionally, the system 500 may be configured to send a message in response to a detected change in acquired medical images. For example, the system 500 may be configured to send a message in response to changes detected in an MR image over time.

The system 500 may be configured to send a message comprising any suitable information about the patient and/or imaging performed on the patient. In some embodiments, system 500 may be configured to send a message comprising one or multiple images produced by the medical imaging device. System 500 may be configured to send, alternately or additionally, metadata associated with the acquisition of the images by the medical imaging device.

In some embodiments, the metadata may comprise information about the patient. For example, the metadata may comprise information about the patient's current vital signs. The metadata may further comprise, for example, information about what condition the patient is being treated for.

In some embodiments, the metadata may comprise information about the image acquired by the medical imaging device. For example, the metadata may comprise information about the time when the image was acquired. The metadata may, for example, further comprise information about the imaging process or protocol used to acquire the image (e.g., imaging parameters). The metadata may, for example, further comprise information about the body part of the patient that is present in the image.

In some embodiments, the metadata may comprise information about the medical imaging device. For example, the metadata may comprise information identifying the physical location of the medical imaging device (e.g., the building and/or room). The metadata may, for example, comprise the type and/or model of the medical imaging device.

In some embodiments, the metadata may comprise information about the user of the medical imaging device. For example, the metadata may comprise the user's name. The metadata may, for example, further comprise contact information for the user.

In some embodiments, the metadata may comprise one or more hyperlinks to additional resources for the recipient of the message. For example, the metadata may comprise a hyperlink to an Internet-based medical image viewing software so that the message recipient may view the imaging results in more detail. The metadata may, for example, comprise a hyperlink to an Internet-based software for remote operation of the medical imaging device so that the message recipient may acquire more images.

Additionally or alternatively, in some embodiments the system 500 may be configured to control, in full or in part, operation of the medical imaging device. System 500 may be, for example, configured to control the acquisition of a medical image using the medical imaging device. System 500 may be configured to control the acquisition of a medical image based on input from the user (e.g., the user's selection of imaging protocols or procedures).

In some embodiments, system 500 may be deployed in a same room as the medical imaging device. For example, in some embodiments, the system 500 may be implemented, in whole or in part, by controller 106 and/or computing device 104 of MRI system 100 as described in connection with FIG. 1. In other embodiments, at least a part of system 500 may be implemented by software stored and/or executed remotely (e.g., as part of a cloud computing environment) from the medical imaging device. In yet other embodiments, each component of system 500 may be implemented by software stored and/or executed remotely from the medical imaging device.

The user interface (UI) 504 shown in the illustrative example of FIG. 5 is a user interface that the medical personnel running the medical imaging device may interact with. The UI 504 may be implemented using a web server, which can run on any computing device (iPad, computer workstation, tablet, phone, laptop, etc.). For example, the UI 504 may be run on computing device 104 of MRI system 100 as described in connection with FIG. 1. Alternately, the UI 504 may be run on any suitable console connected to a medical imaging device (e.g., a portable MRI system as described in connection with FIGS. 3A-3C and/or 4).

For example, in the case of a portable MRI system as described herein, in some embodiments, the UI 504 may allow the user to control the MRI system. For example, the user may be able to select imaging protocols and/or pulse sequences using UI 504. The user may be able to create custom imaging sequences and examination processes using UI 504 (e.g., based on a patient's needs, per request of a physician, etc.) The user may further be able to initiate, pause, and/or end an image acquisition process using UI 504.

In some embodiments, the user may use UI 504 to select who may receive notifications from the MRI system (e.g., from among individual recipients or groups of recipients). The UI 504 may further be configured to allow the user to create and store new groups of recipients (e.g., from pre-populated lists of recipients and/or through manual entry of recipient addresses).

The UI 504 may also be configured to display images acquired by the MRI system during an image acquisition process. Alternately or additionally, the UI 504 may be configured to display status messages associated with the MRI system (e.g., error messages, the remaining time to complete an image acquisition process, etc.).

In some embodiments, the UI 504 may display messages sent in reply to the messages sent by system 500. The messages sent in reply may be from one or more recipients of the messages sent by system 500 (e.g., from medical care team members, supervising physicians, etc.). The UI 504 may further be configured to provide the user with a way to engage in real-time messaging (e.g., instant messaging), in some embodiments. Such real-time messaging may enable rapid communication with a remote physician and/or other medical team member. By responding to messages from system 500 or engaging with a real-time messaging system, medical care team members may quickly request from their present location that the user of the medical imaging device perform additional and/or different image acquisition processes on the patient. This request may be made during an image acquisition process.

In some embodiments, the UI 504 may be configured to allow the user to define the recipients of messages from system 500. The user may be able to select from among email addresses, phone and/or pager numbers, and/or groups of such addresses to notify with messages from the system 500. The user may further select which triggering events may cause system 500 to send a message. For example, the user may select that a message be sent at the start of acquiring an image, at the completion of acquiring an image, at periodic time intervals, and/or if the imaging device detects a change in a patient's status. The user may also, using UI 504, initiate the sending of a message at any time through, for example, a user-initiated request 502.

In some embodiments, the user may, using UI 504, select what type of message is sent by system 500. For example, the user may select in UI 504 whether the message may include a medical image acquired by the medical image device. The user may also select what type of metadata may be included in the message using UI 504. For example, the user may select that the message includes one or more pieces of metadata including but not limited to information about the patient (e.g., their health condition), information about the image acquired by the medical imaging device (e.g., time of acquisition, imaging process or protocol used to acquire the image, body part of the patient that is present in the image, etc.), information about the medical imaging device (e.g., the physical location of the medical imaging device), information about the user of the medical imaging device (e.g., the user's name, contact information for the user), and/or one or more hyperlinks to additional resources for the recipient (e.g., a hyperlink to an Internet-based medical image viewing software, a hyperlink to an Internet-based software for remote operation of the medical imaging device).

In some embodiments, UI 504 may pass information (e.g., selections made by the user, etc.) to and from message controller 506, as shown in the illustrative example of FIG. 5. Message controller 506 may be configured to control the medical imaging device (e.g., to start acquisition of an image, to perform selected imaging acquisition procedures, etc.). Alternatively or additionally, message controller 506 may be configured to create and route messages to one or more selected recipients. Message controller 506 may be configured to create and route messages in response to automated triggering events (e.g., the start and/or end of image acquisition, the end of an examination, etc.) and/or in response to a user-initiated request 502.

In some embodiments, the message controller may be implemented using software that runs on a computing device embedded in the medical imaging device (e.g., controller 106 of MRI system 100 of FIG. 1). Alternately, message controller may be implemented using software stored and/or executed remotely (e.g., as part of a cloud computing environment) from the medical imaging device

In some embodiments, the message controller 506 may be configured to control the medical imaging device's hardware, run imaging sequences, run image reconstruction algorithms, and/or run a link to a communication network (e.g., via ETHERNET, Wi-Fi, cellular, etc.). The message controller 506 may be configured to take requests from the user to create messages via a user-initiated request 502 and may be configured to route the messages to external networked servers using the communication network.

In some embodiments, the message controller 506 may be configured to store, create, and/or route messages during and/or after an exam. For example, the message controller 506 may be configured to create and route messages after each image of an imaging sequence is imaged (not shown). The message controller 506 may additionally or alternately be configured to create and route messages after each image sequence is complete. The message controller 506 may additionally or alternately be configured to create and route messages after an entire exam comprising multiple imaging sequences is complete. The message controller 506 may be configured to create and/or route messages in response to one or more of these aforementioned triggering events based on a selection of the user (e.g., through UI 504 as described herein). Alternately or additionally, the message controller 506 may be configured to automatically select which triggering events will trigger the creation and routing of a message from system 500. In some embodiments, in order to comply with privacy laws (e.g., HIPAA), the message controller 506 may remove confidential and/or identifying information about the patient that could compromise the patient's privacy from the message prior to sending the message.

The message controller 506 may also monitor the patient and create and route messages upon a change in the patient's conditions, in accordance with some embodiments of the technology described herein. For example, the message controller 506 may periodically (e.g., every 20 minutes, every hour, etc.) run an imaging sequence to acquire an MRI image of the patient. The message controller 506 may run software to analyze the acquired MRI image and detect changes by comparing the acquired MRI image to a previously acquired MRI image. For example, the message controller 506 may run software that may detect a midline shift in the patient's brain. Exemplary methods for monitoring a patient's condition are presented in U.S. Patent Application Pub. No. 2018/0143281 filed Nov. 21, 2017 and titled “Systems and Methods for Automated Detection in Magnetic Resonance Images” and U.S. Patent Application Pub. No. 2019/0033415 filed Aug. 29, 2018 and titled “Systems and Methods for Automated Detection in Magnetic Resonance Images,” which are herein incorporated by reference in their entirety. Upon detection of a change in the patient's status, the message controller 506 may create and route a message indicated said status change to one or more members of the medical care team.

The email server 508 shown in the illustrative example of FIG. 5 may be a server running any suitable external networked messaging service, like GMAIL, OUTLOOK, FACETIME, GOOGLE HANGOUTS, SKYPE, etc. Alternately, the email server 508 may be run on a controller associated with the medical imaging device, such as, for example controller 106 as described in connection to FIG. 1. The message controller 506 may route messages through the email server 508. From the email server 508, the messages may be routed to recipient computing devices 510 belonging to members of the medical care team (e.g., physicians, nurses, etc.). Members of the medical care team may also reply back through the external services. The reply may be routed through the message controller 506, which may determine how to display the notification back to the user on the UI 504. The notification could be a ‘received’ confirmation symbol, messages, audio or camera data, or commands to control the interface remotely.

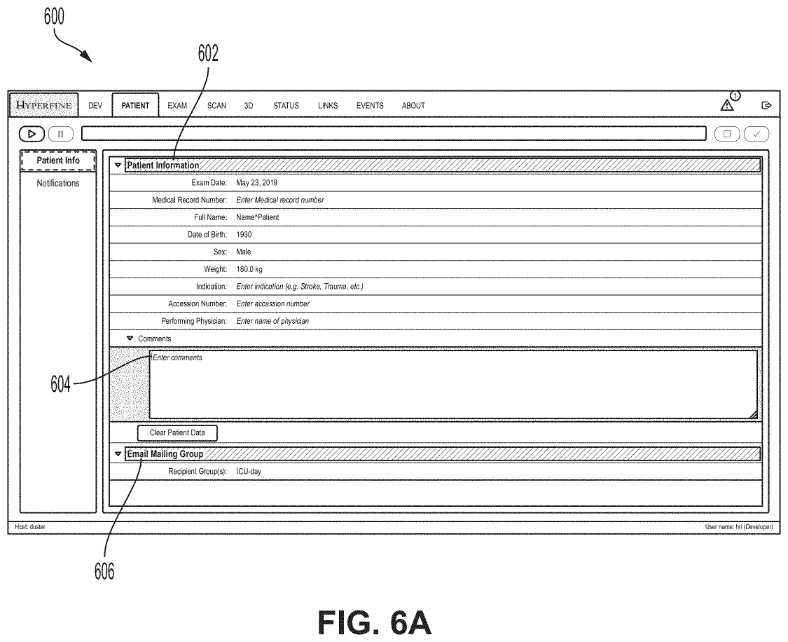

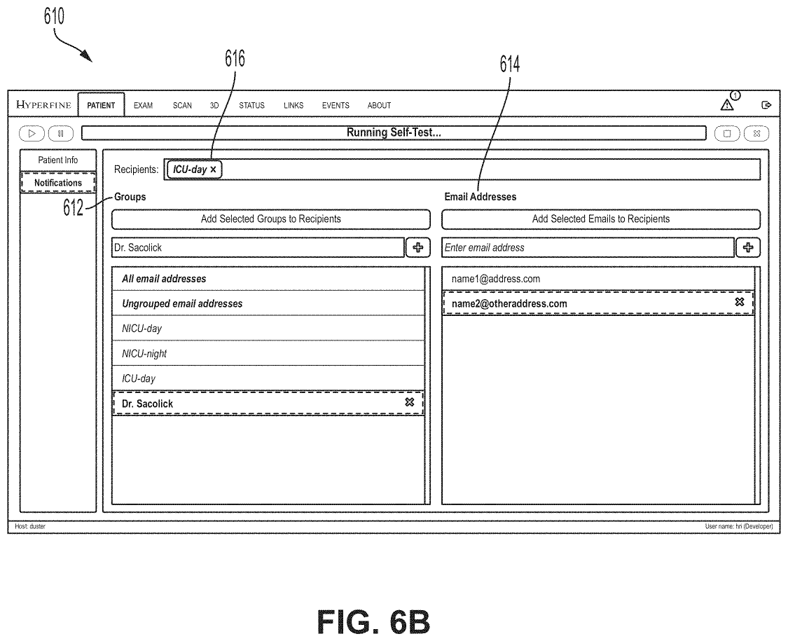

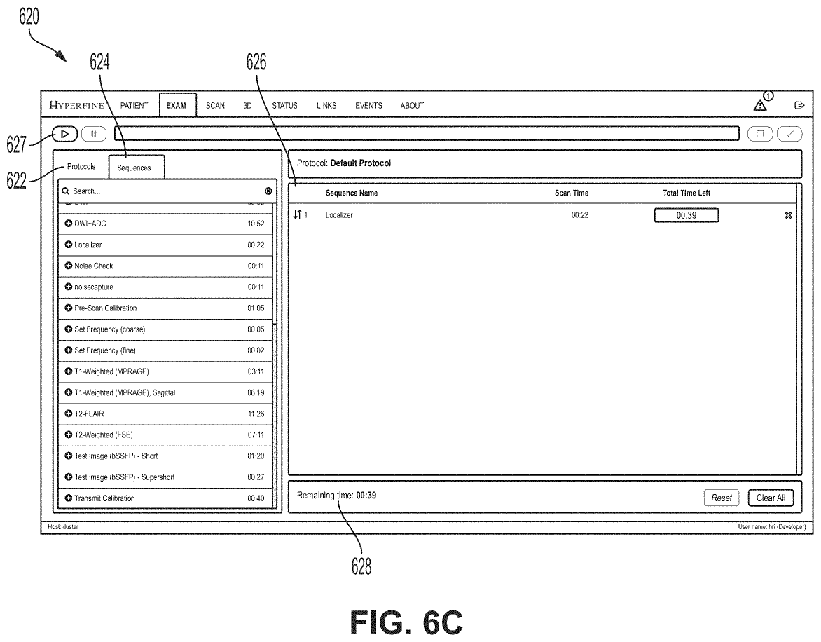

FIG. 6A is an illustrative UI screen 600 for the displaying and entering of patient information, in accordance with some embodiments of the technology described herein. UI screen 600 may be displayed as a part of, for example, UI 504. UI screen 600 may include a section 602 displaying patient information (e.g., name, date of birth, medical condition, etc.) as well as information about the exam procedure (e.g., date of exam, ordering physician, etc.). UI screen 600 may include a section 604 that allows the user to enter comments about the patient and/or procedure. UI screen 600 may further include a section 606 indicating the message recipients. In the example of FIG. 6A, the message recipient is a mailing group for the intensive care unit day shift (ICU-day). However, multiple mailing groups and/or individual addresses may appear in section 606. In some embodiments, a user may also select message recipients in section 606.

FIG. 6B is an illustrative UI screen 610 for the selection and creation of mailing groups and/or individual recipients, in accordance with some embodiments of the technology described herein. UI screen 610 may be displayed as a part of, for example, UI 504. UI screen 610 may include a section 612 displaying available mailing groups that may be selected as recipients for the messaging system. Additionally, UI screen 610 may include a section 614 displaying individual mailing addresses that may be selected as recipients for the messaging system. Section 614 may also allow the user to create custom mailing groups by selecting individual mailing addresses.

Once selected by the user, the selected mailing groups and/or individual addresses may be displayed in section 616. In the example of FIG. 6B, the recipient shown in section 614 includes the mailing group for the intensive care unit day shift (ICU-day). However, multiple mailing groups and/or individual addresses may appear in section 616.

FIG. 6C is an illustrative UI screen 620 for the selection of imaging sequences and protocols, in accordance with some embodiments of the technology described herein. UI screen 620 may be displayed as a part of, for example, UI 504. UI screen 620 may include a section with tabs 622 and 624 for the selection of pre-defined protocols and sequences for the MRI system. In the example of FIG. 6C, the sequences tab 624 is selected and available sequences are listed below the tab 624. However, the user may select protocols from the protocols tab 622 in order to create custom imaging sequences.

When the user selects a sequence and/or protocol from the tabs 622 and/or 624, the sequence and/or protocol may appear in listing 626 along with the estimated time the sequence and/or protocol may take to perform. The user may run the selected sequences and/or protocols shown in the listing 626 by selecting the play button 627, whereupon a total remaining time for the sequences and/or protocols may be shown in section 628. The remaining time for an individual sequence and/or protocol may be shown in the listing 626.

As an exam proceeds and messages are sent to the mailing group(s) and/or individual recipients, feedback may be received from the recipients (e.g., via email server 508 of FIG. 5). The feedback may include requests for additional or alternative imaging sequences and/or protocols to be performed on the patient before the exam concludes. The user may use UI screen 620 to add the requested additional imaging sequences and/or protocols to the exam in real time.

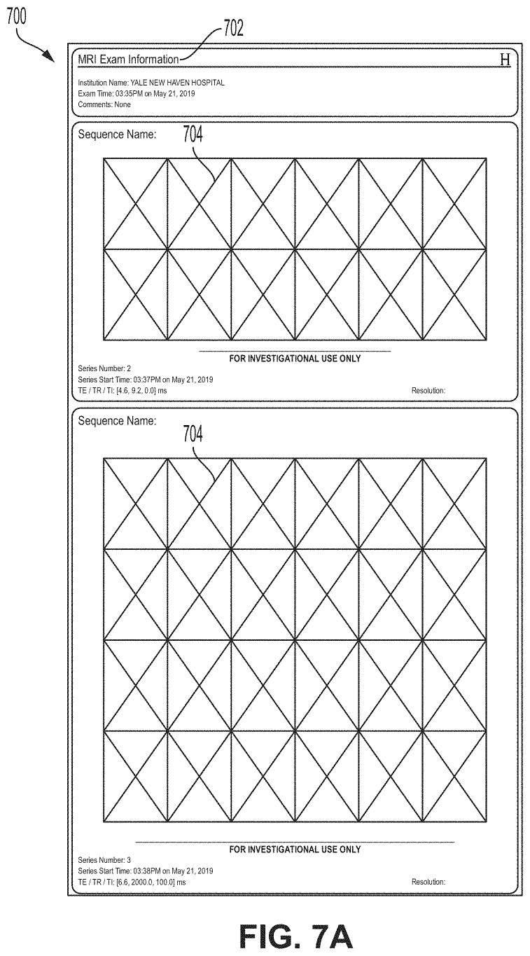

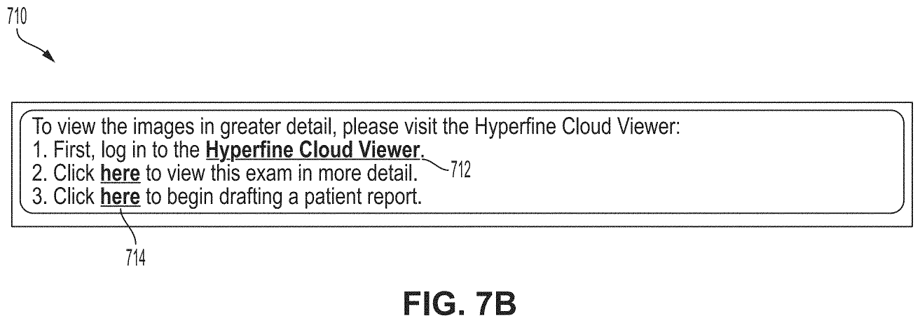

FIG. 7A is an illustrative message 700 sent by, for example, messaging system 500, in accordance with some embodiments of the technology described herein. Message 700 may be sent after the conclusion of an imaging sequence, after the conclusion of an exam, and/or while monitoring a patient, for example. Message 700 may include metadata 702 about the MRI exam (e.g., information about the physical location of the exam, date and time of the exam, and/or comments from the MRI system user).

Message 700 may also include images 704 from an imaging sequence and/or protocol, in accordance with some embodiments of the technology described herein. Images 704 may be accompanied with metadata about the imaging sequence and/or protocol such as the sequence and/or protocol name, the time the imaging sequence and/or protocol was started, and/or the magnetic resonance image resolution.

FIG. 7B is an illustrative message 710 sent by, for example, messaging system 500, in accordance with some embodiments of the technology described herein. Message 710 may be included with message 700 or may be sent separately. Message 710 may include one or more hyperlinks to additional, Internet-based resources. For example, message 710 may include a hyperlink 712 which routes to an Internet-based viewing program. In the example of FIG. 7B, the Internet-based viewing program is the “Hyperfine Cloud Viewer.” The Internet-based viewing program may provide the recipient with more detailed view of the exam results. Message 710 may further include a hyperlink 714 which routes to an Internet-based program for drafting a patient report about the magnetic resonance imaging results.

A recipient of messages 700 and/or 710 may be able to reply to said messages in order to communicate with the user of the MRI system, in accordance with some embodiments of the technology described herein. By replying to messages 700 and/or 710, a recipient of messages 700 and/or 710 may be able to request further imaging sequencing and/or protocols in real time for the user of the MRI system to perform.

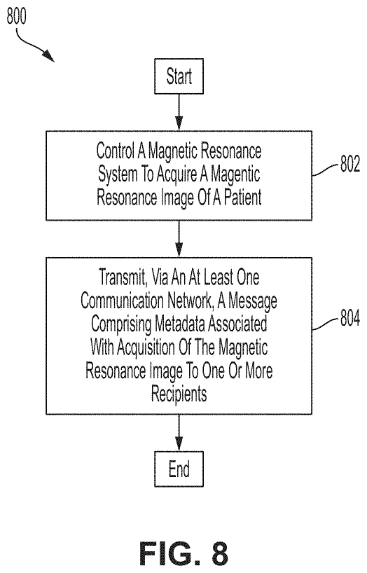

FIG. 8 shows an illustrative process 800 for automatically transmitting messages, in accordance with some embodiments of the technology described herein. For instance, the process 800 may be performed by system 500 described with reference to FIG. 5. In some embodiments, the process 800 may be performed by hardware (e.g., using an ASIC, an FPGA, or any other suitable circuitry), software (e.g., by executing the software using a computer processor), or any suitable combination thereof.

In act 802, a magnetic resonance system may be operated to acquire at least one magnetic resonance image of a patient. The magnetic resonance system may be operated using a controller such as, for example, controller 106 described with reference to FIG. 1. Alternately, in some embodiments, the magnetic resonance system may be operated by, for example, message controller 506 described with reference to FIG. 5. The controller 106 and/or message controller 506 may receive instructions for operating the magnetic resonance system from a user via a UI such as UI 504. Received instructions may include which imaging sequences and/or protocols the magnetic resonance system should perform.

In some embodiments, the magnetic resonance system may be, for example, a low-field and/or portable magnetic resonance imaging system as described with reference to FIGS. 2, 3A-3C, and/or 4. The controller may be located in the same room as the magnetics system of the magnetic resonance system and may be communicatively coupled to a communication network (e.g., via Ethernet, Wi-Fi, etc.) in order to transmit messages.

Next, process 800 proceeds to act 804, where a message may be communicated via the communication network to one or more recipients. The recipients may be specified by the user of the magnetic resonance system prior to acquiring the at least one magnetic resonance image of the patient. The recipients may be specified individually (e.g., by specifying individual addresses) or by selecting recipient groups (e.g., selecting a medical care team associated with the patient).

In some embodiments, the message (e.g., an email, a short message service (SMS), a multimedia message service (MMS), etc.) may contain metadata associated with the acquisition of the magnetic resonance image. The metadata may be any information associated with the acquisition of the magnetic resonance image. For example, the metadata may include information about the physical location of the magnetic resonance system, information identifying the user of the magnetic resonance system and/or the user's contact information, information about the patient, information about the imaging protocol and/or sequence used, etc. Additionally, the metadata may also include hyperlinks to web-based applications such as magnetic resonance image viewing software program and/or a program for remote operation of the magnetic resonance system. Prior to transmitting the message, confidential and/or identifying information about the patient may be removed from the message.

In some embodiments, transmittal of the message may be triggered by different triggering events. The triggering events may include the completion of an imaging sequence or protocol or the completion of an entire examination including multiple imaging sequences and/or protocols. Alternatively, transmittal of the message may be triggered by the user of the magnetic resonance system at any time during the examination. When monitoring a patient over a period of time, transmittal of the message may be triggered by a detected change or changes in the acquired magnetic resonance images.

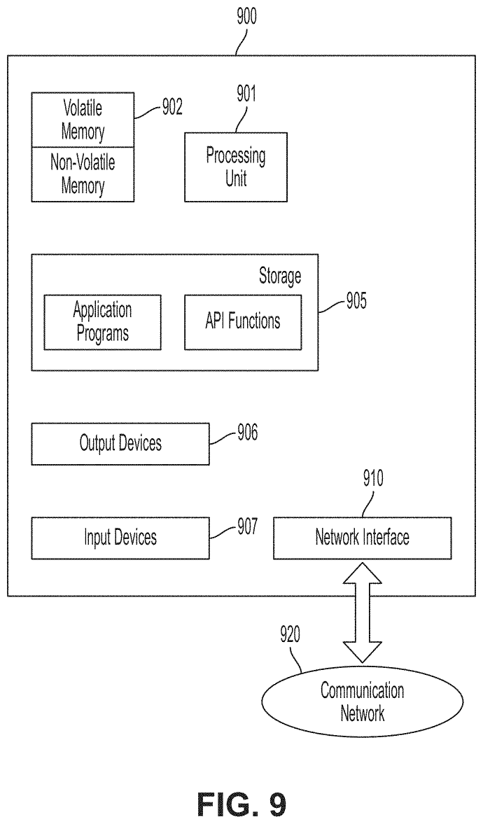

FIG. 9 shows, schematically, an illustrative computer 900 on which any aspect of the present disclosure may be implemented. In the embodiment shown in FIG. 9, the computer 900 includes a processing unit 901 having one or more processors and a non-transitory computer-readable storage medium 902 that may include, for example, volatile and/or non-volatile memory. The memory 902 may store one or more instructions to program the processing unit 901 to perform any of the functions described herein. The computer 900 may also include other types of non-transitory computer-readable medium, such as storage 905 (e.g., one or more disk drives) in addition to the system memory 902. The storage 905 may also store one or more application programs and/or resources used by application programs (e.g., software libraries), which may be loaded into the memory 902.

The computer 900 may have one or more input devices and/or output devices, such as devices 906 and 907 illustrated in FIG. 9. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, the input devices 907 may include a microphone for capturing audio signals, and the output devices 906 may include a display screen for visually rendering, and/or a speaker for audibly rendering, recognized text. As another example, the input devices 907 may include sensors (e.g., electrodes in a pacemaker), and the output devices 906 may include a device configured to interpret and/or render signals collected by the sensors (e.g., a device configured to generate an electrocardiogram based on signals collected by the electrodes in the pacemaker).

As shown in FIG. 9, the computer 900 may also comprise one or more network interfaces (e.g., the network interface 910) to enable communication via various networks (e.g., the network 920). Examples of networks include a local area network or a wide area network, such as an enterprise network or the Internet. Such networks may be based on any suitable technology and may operate according to any suitable protocol and may include wireless networks, wired networks or fiber optic networks. Such networks may include analog and/or digital networks.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the present disclosure. Further, though advantages of the concepts described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semi-custom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the various methods or processes outlined herein may be coded as software that is executable on one or more processors that employ any one of a variety of operating systems or platforms. However, it should be appreciated that aspects of the present disclosure are not limited to using an operating system. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure described above. The computer-readable medium or media may be transportable, such that the program or programs stored thereon can be loaded onto one or more different computers or other processors to implement various aspects of the present disclosure as described above.

The terms “program” or “software” are used herein to refer to any type of computer code or set of computer-executable instructions that can be employed to program a computer or other processor to implement various aspects of the present disclosure as described above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the concepts disclosed herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which one or more examples has been provided, including, for example, with reference to FIG. 8. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A magnetic resonance imaging (MRI) system, comprising:
a magnetics system having a plurality of magnetics components configured to produce magnetic fields to perform MRI; and
a controller, communicatively coupled to the magnetics system and at least one communication network, and configured to:
control the magnetics system to acquire a magnetic resonance (MR) image of a patient; and
in response to a triggering event,
transmit, via the at least one communication network, a message comprising metadata associated with acquisition of the MR image and/or the MR image to one or more recipients.

2. The MRI system of claim 1, wherein the controller is located in a same room as the magnetics system.

3. The MRI system of claim 1, wherein the metadata associated with acquisition of the MR image comprises at least one of:
information about the patient;
information about the MRI protocol associated with acquisition of the MR image;
information identifying an operator of the MRI system and/or contact information associated with the operator;
information identifying the physical location of the MRI system;
a hyperlink to a web-based MR image viewing software program; and
a hyperlink to an interface for remote operation of the MRI system.

4. The MRI system of claim 1, wherein the triggering event comprises at least one of input received from an operator of the MRI system, completion of acquisition of the MR image, and start of acquisition of the MR image.

5. The MRI system of claim 1, wherein the magnetics system comprises a B₀ magnet comprising a permanent magnet.

6. The MRI system of claim 1, wherein the magnetics system comprises a B₀ magnet configured to produce a B₀ magnetic field having a field strength equal to or less than approximately 0.2 T and greater than or equal to approximately 20 mT.

7. The MRI system of claim 1, wherein the magnetics system comprises a B₀ magnet configured to produce a B₀ magnetic field having a field strength equal to or less than approximately 1 T and greater than or equal to approximately 50 mT.

8. The MRI system of claim 1, wherein the magnetics system comprises a B₀ magnet configured to produce a B₀ magnetic field having a field strength greater than or equal to approximately 1 T.

9. The MRI system of claim 1, further comprising a conveyance mechanism to allow the MRI system to be moved to different locations.

10. A method of operating a magnetic resonance imaging (MRI) system, the MRI system comprising a magnetics system having a plurality of magnetics components configured to produce magnetic fields to perform MRI, the method comprising:
using a controller communicatively coupled to at least one communication network to:
control the MRI system to acquire a magnetic resonance (MR) image of a patient; and
in response to a triggering event:
transmit, via the at least one communication network, a message comprising metadata associated with acquisition of the MR image and/or the MR image to one or more recipients.

11. The method of claim 10, wherein the controller is located in a same room as the magnetics system.

12. The method of claim 10, wherein transmitting the message comprises transmitting one of an email, a short message service (SMS), and/or a multimedia messaging service (MMS).

13. The method of claim 10, further comprising removing confidential patient information from the metadata associated with acquisition of the MR image prior to transmitting the message.

14. The method of claim 10, wherein transmitting the message comprising metadata associated with acquisition of the MR image comprises transmitting a message comprising one or more of:
information about the patient;
information about the MRI protocol associated with acquisition of the MR image;
information identifying an operator of the MRI system and/or contact information associated with the operator;
information identifying the physical location of the MRI system;
a hyperlink to a web-based MR image viewing software program; and
a hyperlink to an interface for remote operation of the MRI system.

15. The method of claim 10, wherein the triggering event comprises one of input received from an operator of the MRI system, completion of acquisition of the MR image, and start of acquisition of the MR image.

16. At least one non-transitory computer-readable storage medium storing processor-executable instructions that, when executed by a magnetic resonance imaging (MRI) system, cause the MRI system to perform a method comprising:
using a controller communicatively coupled to at least one communication network to:
control the MRI system to acquire a magnetic resonance (MR) image of a patient; and
in response to a triggering event:
transmit, via the at least one communication network, a message comprising metadata associated with acquisition of the MR image and/or the MR image to one or more recipients.

17. The at least one non-transitory computer-readable storage medium of claim 16, wherein the MRI system comprises a magnetics system; and
the controller is located in a same room as a magnetics system.

18. The at least one non-transitory computer-readable storage medium of claim 16, wherein the message comprises an email, a short message service (SMS), and/or a multimedia messaging service (MMS).

19. The at least one non-transitory computer-readable storage medium of claim 16, wherein the metadata associated with acquisition of the MR image comprises one or more of:
information about the patient;
information about the MRI protocol associated with acquisition of the MR image;
information identifying an operator of the MRI system and/or contact information associated with the operator;
information identifying the physical location of the MRI system;
a hyperlink to a web-based MR image viewing software program; and
a hyperlink to an interface for remote operation of the MRI system.

20. The at least one non-transitory computer-readable storage medium of claim 16, wherein the triggering event comprises one of input received from an operator of the MRI system, completion of acquisition of the MR image, and start of acquisition of the MR image.
